(19) United States
(12) Patent Application Publication
(10) Pub. No.: US 2019/0194236 A1
(43) Pub. Date: Jun. 27, 2019

(54) Organometallic Antibiotics

(71) Applicant: Ian Michael Henderson, Edgewood, NM (US)

(72) Inventor: Ian Michael Henderson, Edgewood, NM (US)

(21) Appl. No.: 15/851,696

(22) Filed: Dec. 21, 2017

(51) Int. Cl. C07F 15/00; A61P 31/00; A61P 31/04

(52) U.S. Cl. CPC C07F 15/0086; A61P 31/04; A61P 31/00

(57) ABSTRACT

The present invention discloses a class of antibiotics designed to target non-protein transporters. Further, a method is described to treat infections by targeted binding of a lipid transporter utilizing organometallic compounds, coordination compounds, metal centers, or any combination thereof. The antibiotics described herein are designed to halt or negatively affect the reproduction of bacteria by disrupting the biosynthesis of the cell wall and other important bacterial compounds such as lipopolysaccharide and techoic acids.

### REFERENCE TO RELATED APPLICATIONS

This application claims an invention which was disclosed in Provisional Application No. 62/442,162, filed Jan. 4, 2017, entitled, “Terpenoid-Targeting Antibioitcs”. The benefit under 35 USC § 119(e) of the United States provisional application is hereby claimed, and the aforementioned application is hereby incorporated herein by reference.

### FIELD OF THE INVENTION

The invention pertains to a novel class of antibiotics designed to target non-protein transporters.

### BACKGROUND OF THE INVENTION

The disruption of the biosynthesis of bacterial cell wall components is a proven antibiotic mechanism of action. Difficulties often arise with the binding of antibiotics to the proteins involved in cell-wall biosynthesis and production of various other important bacterial compounds, as the protein-based molecular targets involved in the biosynthetic process can often undergo target alteration, which inactivates antibiotic compound while maintaining biosynthetic processes.

In lieu of a protein target, some antibiotics such as bacitracin and the recently discovered teixobactin target non-protein transporters within the bacterial cell. These antibiotics have proven effective against gram positive bacteria, but have shown little promise against gram-negative species. The limitations of current transporter-binding antibiotics, along with the general lack of effective treatments for gram-negative infections, make evident the need for an effective antibiotic capable of binding to lipid transporters and being capable of crossing the gram-negative cell membrane.

### SUMMARY OF THE INVENTION

The limitations of current transporter-binding antibiotics, along with the general lack of effective treatments for gram-negative infections, make evident the need for an effective antibiotic capable of binding to lipid transporters and being capable of crossing the gram-negative cell membrane. Herein, a novel class of antibiotics designed to target non-protein transporters is described. The antibiotics described herein are designed to kill or slow the reproduction of bacteria by disrupting the biosynthesis of the cell wall and other important bacterial compounds such as lipopolysaccharide and techoic acids.

A chemical compound having the formula shown in FIG. 1, wherein R¹ is a hydrophobic group comprising an aryl, alkyl, or flouoalkyl moiety; wherein R² and R³, which are the same or different and are hydrogen, halogen, CR₃, CF₃, or part of an aromatic ring; wherein R⁴ is a chemical group capable of binding a phosphate or pyrophosphate, comprising hydrogen bond donors, groups bearing a positive charge, or groups capable of phosphate and/or pyrophosphate binding; wherein x and y are the same or different and are 0-6; wherein M is a metal selected from Pt, Pd, or Ni, having oxidation states from 0 to (IV); wherein L¹ and L² are the same or different and are selected from alkenes or phosphines.

The chemical compound of claim 1 wherein L¹ and L² further bind to Z such to have a configuration as shown in FIG. 2C; wherein Z is a bridging or linking group of 0-10 atoms in length comprising C, Si, 0, or N atoms with any variation.

A method of treating infections, broadly defined, via targeted binding of a lipid transporter utilizing organometallic compounds, coordination compounds, metal centers, or any combination thereof. The method of treating infections of claim 3 wherein the target organisms are gram-positive bacteria. The method of treating infections of claim 3 wherein the target organisms are gram-negative bacteria. The method of treating infections of claim 3 wherein the target organisms are mycobacteria. The method of treating infections of claim 3 wherein the target organisms are pathogens other than bacteria. The method of treating infections of claim 3 wherein the target molecule is an isoprenoid and/or terpenoid.

A method of treating infections, broadly defined, via targeted binding of isoprenoid phosphates, and isoprenoid and/or terpenoid compounds other than pyrophosphate derivatives and isoprenoid-based lipids containing a pyrophosphate linker. The method of treating infections of claim 9 wherein the target organisms are gram-positive bacteria. The method of treating infections of claim 9 wherein the target organisms are gram-negative bacteria. The method of treating infections of claim 9 wherein the target organisms are mycobacteria. The method of treating infections of claim 9 wherein the target organisms are pathogens other than bacteria.

### BRIEF DESCRIPTION OF THE DRAWING

FIG. 1 shows an example embodiment of a parent chemical formula of an organometallic antibiotic.

FIG. 2A shows an example embodiment of chemical formula of R⁴

FIG. 2B shows another example embodiment of chemical formula of R⁴

FIG. 2C shows an example embodiment of a linking group Z

FIG. 3 shows an example embodiment of 7 different organometallic antibiotics arising from the parent chemical formula of FIG. 1

FIG. 4 shows ligand dissociation and coordination to undecaprenyl phosphate.

FIG. 5A shows Zone of inhibition (ZOI) studies with Compound 1 for S. Areus (left) and S. Epidermidus (right).

FIG. 5B shows mouse 3T3 fibroblasts treated with 25 μg/ml of Compound 1.

FIG. 6A depicts the effects of various concentrations of 1 administered to cultures of K12 E. coli.

FIG. 6B depicts the effects of various concentrations of 1 administered to cultures of S. areus.

FIG. 6C depicts the effects of various concentrations of 1 administered to cultures of S. Epidermidus

FIG. 7 shows a chemical equation for the synthesis of the ligand “B-TAID”.

FIG. 8 shows a chemical equation of the synthesis of compound 1 is shown in Formula 3

### DETAILED DESCRIPTION OF THE INVENTION

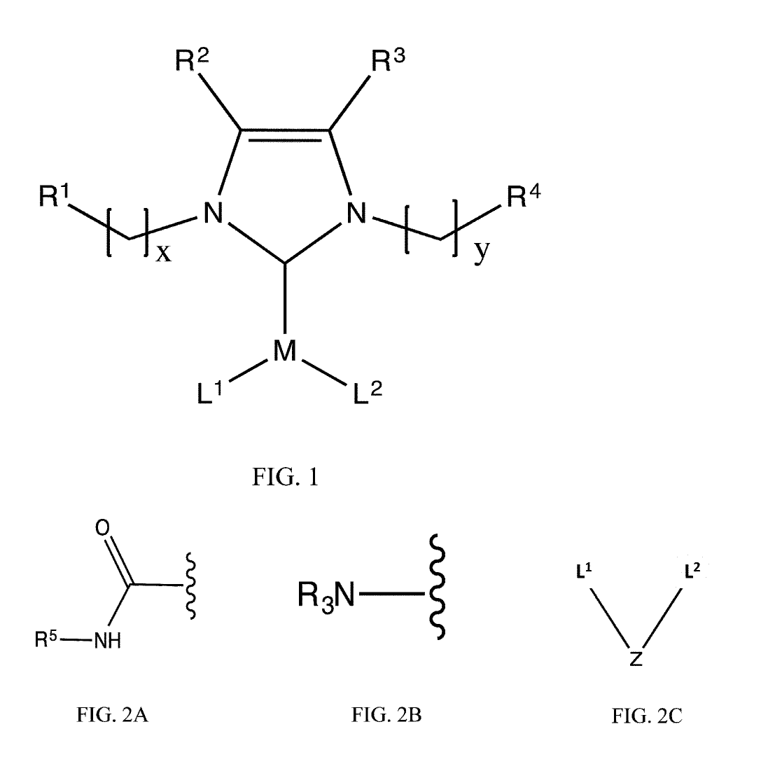

As shown in FIG. 1, R¹ is a hydrophobic group comprising an aryl, alkyl, or flouoalkyl moiety. The capability of binding to the phosphate or pyrophosphate end-groups of isoprenoid transporters is one of three associations that align and bind the drug to the transporter. To accomplish this binding motif, R¹ is selected from either positively charged or hydrogen-bond donating groups that are capable of binding to phosphate or pyrophosphate. These include, but are not limited to the following: ammonium ions, primary and secondary amides, phenols, sulfamides, etc.

R² and R³, which are the same or different and are hydrogen, halogen, CR₃, CF₃, or part of an aromatic ring. R² and R³ are independent from each other. R⁴ is a chemical group capable of binding a phosphate or pyrophosphate, comprising hydrogen bond donors, groups bearing a positive charge, or groups capable of phosphate and/or pyrophosphate binding. The groups R² and R³ may be hydrogen atoms, halides, alkyls, aryls, or part of a fused aryl ring. Changing the composition of these groups is used to change the binding capabilities of the metal center to the isoprenoid, has well as to modulate hydrophilicity and other factors. The hydrogen bond donors can be amides, ureas, or protonated amines and guanidine; groups bearing a positive charge can be fully alkylated amines.

A second interaction by which the drug interacts with the isoprenoid transport is a hydrophobic interaction. This interaction is accomplished by various embodiments of functional group R⁴. The R⁴ group may include several different permutation of aryl, alkyl, haloalkyl, haloaryl, or any other of a number of various hydrophobic functional groups. In one embodiment of the invention, R⁴ can be a hydrogen bond donating group such as an amide according to FIG. 2A. In another example embodiment, R₄ can be a positively-charged group such as the ammonium groups shown in FIG. 2B wherein the group “R” in FIG. 2B may be any combination of alkyl groups.

The third mechanism by which the drug binds to isoprenoid transporters is the interaction of the metal center with the alkene bonds in the isoprenoid backbone. The metal center, “M” is a metal selected from the Group 10 metals Pt, Pd, or Ni, having oxidation states from 0 to (IV). This metal is bound to L₁ and L₂. L₁ and L₂ are the same or different and are selected from alkenes or phosphines binding M to various degrees, including linked groups such that L₁ and L₂ is linked to Z as seen in FIG. 2C wherein Z is a bridging or linking group of 0-10 atoms in length containing C, Si, O, or N atoms with any variation of substituent groups. It is noted that each occurrence of alkyl, alkenyl, alkynyl is branched or unbranched, and aryl groups may be substituted or unsubstituted in FIG. 1. This invention further provides a pharmaceutical composition comprising a parent compound of FIG. 1 and a suitable carrier.

The linkers x and y may be the same or different and may range from 0-6. Linking groups may be substituted to adjust for the best possible target binding. The numbers here indicate how many times x and y can occur within the parent chemical formula in FIG. 1. It is also important to note that x and y are independent from each other.

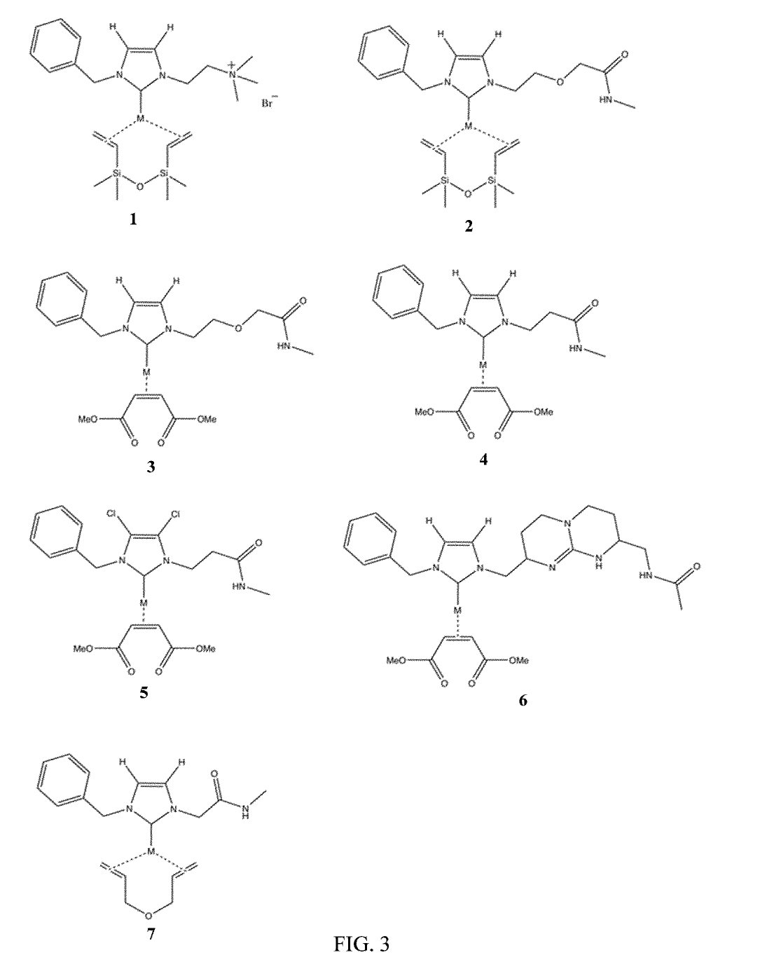

7 different non-limiting examples of the class of antibiotics embodied in the invention is shown in FIG. 3. In one embodiment, compound 1 was synthesized according to a two-step process.

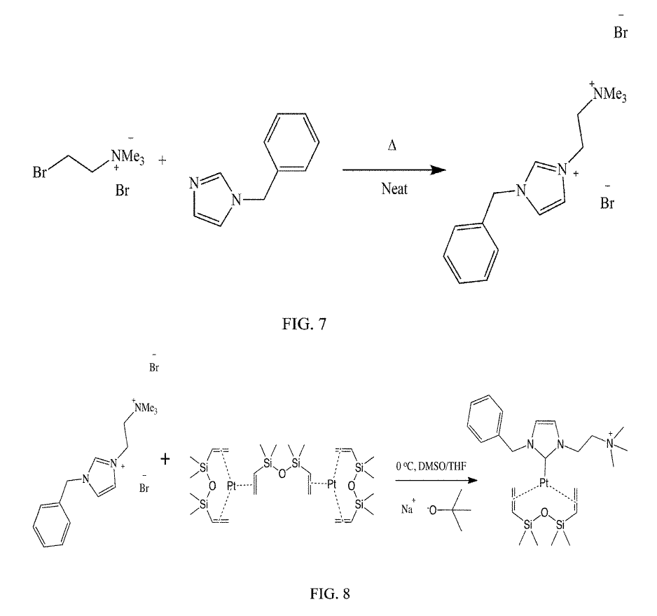

FIG. 7 shows the summary chemical equation for the synthesis of ligand “B-TAID” (1-benzyl-3-(2-trimethylammonium)ethyl imidazolium dibromide. The synthesis of ligand “B-TAID” is as follows:

To a 50 ml round-bottomed flask was added 0.6 g of bromoethyltrimethylammonium bromide, 1.5 g of 1-bezylimidazole and a stirbar. The flask was evacuated and backfilled with nitrogen times, and heated in an 85° C. for 16 h with stirring. After this time the septum was removed and 10 ml of ethanol was added to the reaction mixture and allowed to boil off over the next 5 h. After this, 10 ml THF was added to the flask and allowed to boil for 5 min with stirring. The flask was allowed to cool, the THF decanted, and the process repeated with toluene, then again with THF. The crude product was then taken up in 3 ml water and filtered into 25 ml of THF. To this mixture was added 10 ml toluene. Upon separation of the phases, the bottom phase was removed and evaporated to dryness under high vacuum. Yield: 0.83 g (83.3%) H¹ NMR (90 MHz, D₂O) δ=8.0-7.0, (7H, aromatic/imidazole), 5.510 (2H, benzyl CH₂), 4.808 (2H, ethyl), 4.094 (2H, ethyl), 3.414 (9H, trimethylammonium).

FIG. 8 shows a summary chemical equation of the synthesis of compound 1 from B-TAID. The synthesis of compound 1 from B-TAID is as follows:

To a 50 ml round-bottom flask was added 0.25 g of B-TAID and 0.6 g of Karstedt's catalyst (20% Pt). The mixture was placed under vacuum to remove volatiles. After this 1 ml of anhydrous DMSO was added to dissolve the B-TAID, followed by 1 ml of anhydrous THF. The homogenous mixture was sparged with nitrogen for 5 min before the flask containing the mixture was cooled in an ice bath for 20 min. After this 32 mg of sodium tert-butoxide was added to the reaction mixture. The mixture was stirred under nitrogen before a second 32 mg fraction of sodium tert-butoxide was added. After 20 minutes of stirring, the mixture solidified and was removed from the ice bath, and allowed to stir 19 h under nitrogen at room temperature. The mixture was added to 40 ml of toluene, yielding an off-white precipitate (65 mg). The product was further purified on a diatomaceous earth column (silica gel degrades the product) using 10:1 methylene chloride as an eluent. H¹ NMR (90 MHz, CDCl₃) δ=8.5-6.5 (7H, aromatic/imidazole), 5.179 (2H, benzyl CH₂), 4.670 (2H, ethyl), 4.370 (2H, ethyl), 3.456 (9H, trimethylammonium), 2.0-0.5 (6H, ligand vinyl), 0.375 (6H, ligand dimethyl), −0.338 (6H, ligand dimethyl) Pt¹⁹⁵ NMR (19.42 MHz, CDCl₃) δ=−5334.396

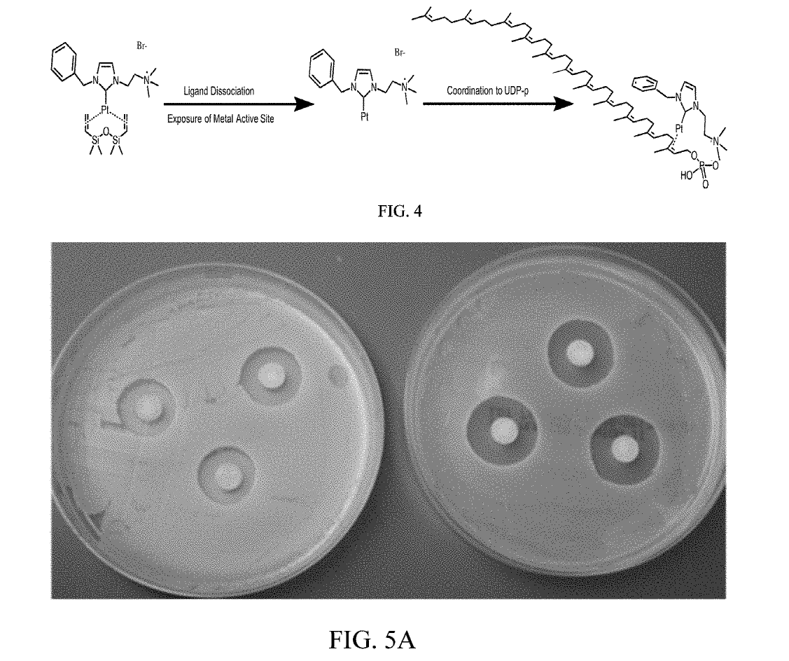

This invention provides a method of stopping bacterial growth and/or killing bacteria using the above compounds and/or a pharmaceutical mixture and/or pharmaceutically acceptable salt thereof. One example of doing so is through the method of binding to and deactivating lipid-based carriers within the bacterial cell. In an example embodiment (FIG. 4), ligand dissociation occurs exposing the metal active site of compound 1 of FIG. 3 which is the M in the parent chemical formula in FIG. 1. This exposure allows for the antibiotic chemical compound to have coordination to (undecaprenyl phosphate) UDP-p. The invention provides a method of binding to isoprenoids, a subset of which serves as bacterial transporters, in a directed fashion, so as to prevent the binding of carbohydrates to the isoprenoid transporters.

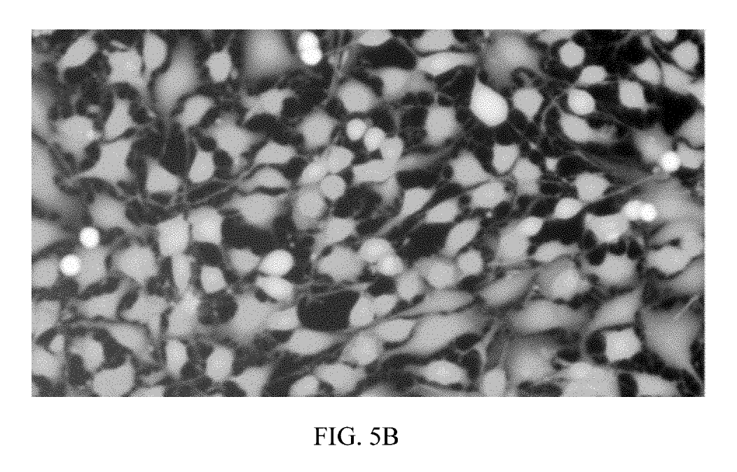

As shown in FIG. 5A, Zone of Inhibition (ZOI) studies with Compound 1 for S. Areus (left) and S. Epidermidus (right). The loading was 33 μg/disk, and the ZOIs were 17.2±0.6 mm and 14.4±0.5, respectively. While in FIG. 5B, mouse 3T3 fibroblasts treated with 25 μg/ml of Compound 1. The cells were assayed calcein-AM/ethidium iodide live/dead assay, returning and ID₅₀ of between 50 and 25 μg/ml.

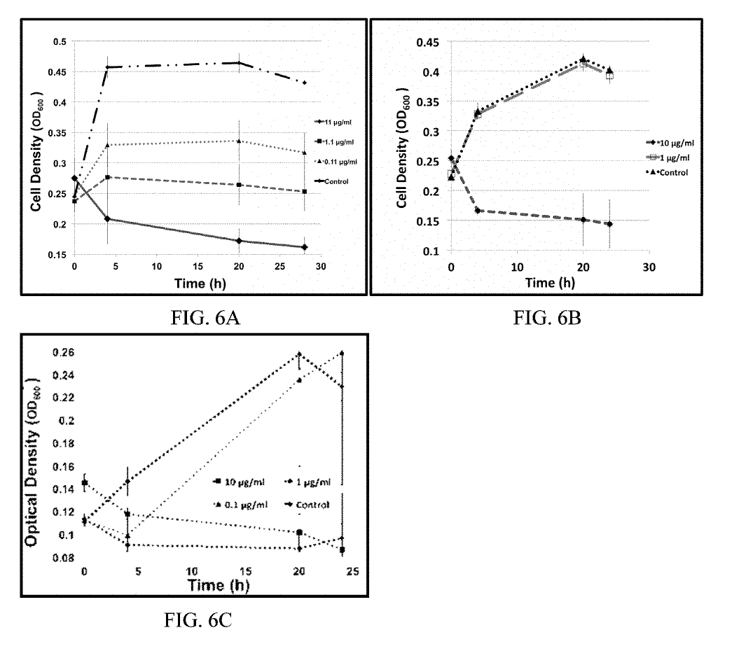

As shown in FIG. 6A, addition of Compound 1 to a culture of K12 E. Coli returned a minimum inhibitory concentration (MIC) of 1.1 μg/ml. Meaning that at this concentration, no growth of the bacteria was observed. Growth was slowed, but not eliminated for the cultures treated with 0.11 μg/ml, and bacterial death (negative growth) was observed in the 11.1 μg/ml sample. As shown in FIG. 6B, for cultures of S. Areus treated with Compound 1, the 1 μg/ml sample showed no change from the, while the 10 μg/ml sample clearly showed negative growth. A follow-up study indicated an MIC values of ≥7 μg/ml. It should be noted that, in the case of S. Areus, concentrations above the MIC were very fast-acting, showing depletion of the cell density within 4 hours. As shown in FIG. 6C, for cultures of S. Epidermidus treated with Compound 1, the 0.1 μg/ml sample showed no change from the, while the 1 μg/ml and 10 μg/ml sample clearly showed negative growth. This led us to determine an MIC of less than or equal to 1 μg/ml for S. Epidermidus.

The term “group” or “functional group” used to describe a chemical entity that is attached to and/or part of the invention.

The term “organic” or “organic group” as used to describe the invention herein refers to a aliphatic or aromatic hydrocarbon group, which may be linear or cyclic or any combination thereof. The term “organic group” in this context refers to a group having no interference on the function of the invention, and includes alkyl, aryl, alkenyl, and alkynyl groups, linear or branched, saturated or unsaturated, and may also contain heteroatoms.

The prefix “alk”, and the term “alkyl”, includes any hydrocarbon group, straight, cyclic or branched.

The term “haloalkyl” includes any alkyl group substituted with one or more halogen atoms in any position.

The terms “heterocycle”, and ‘heterocyclic’, includes non-aryl cyclic structures with at least one heteroatom (O, S, Si, N, etc.).

The term “heteroaryl” refers to an aromatic structure containing at least one heteroatom.

The term “metal center” includes a metal atom included as part of the invention.

Aryl groups may be substituted or unsubstituted.

The term “group” refers to various chemical atoms, substituents or moieties within or part of the molecules discussed and is used as a means to simplify communication. The term “group” should not be taken to limit the type of atom, substituent, or moiety in either structure or function.

The invention is inclusive of all compounds defined herein, including all salts, solvates, mixtures containing the compounds embodied herein, tautomers, isomers, diastereomers, polymorphs, prodrugs, hydrates, physical and chemical conjugates, and the like. The term “compound” is taken to mean all conceivable permutations of the invention, and include any and all such forms, whether explicitly stated or not. This includes pharmaceutically relevant salts, mixtures, and compositions.

“Treating”, “treatment”, “treat”, etc. as used herein describes the prophylactic and/or therapeutic action intended to mitigate a least one symptom of a patient's condition.

The term “patient” or “subject” as used herein refers to humans, non-human animals, organisms in which a condition is to be treated, etc.

The term “target” as used herein describes the molecule or molecules to which the compounds embodied herein bind in order to affect a treatment of disease.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A chemical compound having the formula: 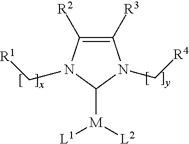
wherein R¹ is a hydrophobic group comprising an aryl, alkyl, or flouoalkyl moiety;
wherein R² and R³, which are the same or different and are hydrogen, halogen, CR₃, CF₃, or part of an aromatic ring;
wherein R⁴ is a chemical group capable of binding a phosphate or pyrophosphate, comprising hydrogen bond donors, groups bearing a positive charge, or groups capable of phosphate and/or pyrophosphate binding;
wherein x and y are the same or different and are 0-6;
wherein M is a metal selected from Pt, Pd, or Ni, having oxidation states from 0 to (IV);
wherein L¹ and L² are the same or different and are selected from alkenes or phosphines.

2. The chemical compound of claim 1 wherein L¹ and L² further bind to Z such that: 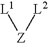
wherein Z is a bridging or linking group of 0-10 atoms in length comprising C, Si, O, or N atoms with any variation.

3. A method of treating infections, broadly defined, via targeted binding of a lipid transporter utilizing organometallic compounds, coordination compounds, metal centers, or any combination thereof.

4. The method of treating infections of claim 3 wherein the target organisms are gram-positive bacteria.

5. The method of treating infections of claim 3 wherein the target organisms are gram-negative bacteria.

6. The method of treating infections of claim 3 wherein the target organisms are mycobacteria.

7. The method of treating infections of claim 3 wherein the target organisms are pathogens other than bacteria.

8. The method of treating infections of claim 3 wherein the target molecule is an isoprenoid and/or terpenoid.

9. A method of treating infections, broadly defined, via targeted binding of isoprenoid phosphates, and isoprenoid and/or terpenoid compounds other than pyrophosphate derivatives and isoprenoid-based lipids containing a pyrophosphate linker.

10. The method of treating infections of claim 9 wherein the target organisms are gram-positive bacteria.

11. The method of treating infections of claim 9 wherein the target organisms are gram-negative bacteria.

12. The method of treating infections of claim 9 wherein the target organisms are mycobacteria.

13. The method of treating infections of claim 9 wherein the target organisms are pathogens other than bacteria.
